(19) United States
(12) Patent Application Publication
(10) Pub. No.: US 2025/0302417 A1
(43) Pub. Date: Oct. 2, 2025

(54) IMAGE STITCHING METHOD FOR X-RAY IMAGING SYSTEM, X-RAY IMAGING METHOD, AND X-RAY IMAGING SYSTEMS

(71) Applicant: GE Precision Healthcare LLC, Waukesha, WI (US)

(72) Inventors: Aizhen Zhou, Beijing (CN); Yingying Wang, Beijing (CN); Dejun Wang, Beijing (CN); Min Liu, Beijing (CN)

(21) Appl. No.: 19/094,901

(22) Filed: Mar. 29, 2025

(30) Foreign Application Priority Data
Mar. 29, 2024 (CN) 202410381073.3

(51) Int. Cl. A61B 6/00; A61B 6/50

(52) U.S. Cl. CPC A61B 6/5241; A61B 6/505; A61B 6/547

(57) ABSTRACT

Provided in the present application are an image stitching method for an X-ray imaging system, an X-ray imaging method, and X-ray imaging systems. The X-ray imaging system includes an X-ray source, and the image stitching method includes dividing an imaging region of a subject under examination into a first region and a second region, the first region and the second region having an overlapping portion; acquiring a first quantity of X-ray images of the subject under examination by rotating the X-ray source in the first region; acquiring a second quantity of X-ray images of the subject under examination by translating the X-ray source in the second region; and performing image stitching on the first quantity of X-ray images and the second quantity of X-ray images to acquire a medical image of the subject under examination.

### CROSS-REFERENCE TO RELATED APPLICATIONS

The present application claims priority to Chinese Application No. 202410381073.3, filed on Mar. 29, 2024, the entire contents of which is hereby incorporated by reference.

### TECHNICAL FIELD

The present invention relates to medical imaging technologies, and more specifically to an image stitching method for an X-ray imaging system, an X-ray imaging method, and X-ray imaging systems.

### BACKGROUND

In an X-ray imaging system, radiation from an X-ray source is emitted toward a subject, and the subject under examination is usually a patient in a medical diagnosis application. Some of the radiation passes through the subject under examination and impacts a detector, which is divided into a matrix of discrete elements (e.g., pixels). The detector elements are read to generate an output signal on the basis of the amount or intensity of radiation that impacts each pixel region. The signal can then be processed to generate a medical image that can be displayed for review, and the medical image can be displayed in a display apparatus of the X-ray imaging system.

During scanning, it is sometimes necessary to acquire a whole-body image of a subject under examination, for example, it may be necessary to determine or view the condition of the lower limbs from the state of the spine, or determine the influence of the lower limbs on the spine from the condition of the lower limbs, or the like.

### SUMMARY

The present invention provides an image stitching method for an X-ray imaging system, an X-ray imaging method, and X-ray imaging systems.

Exemplary embodiments of the present invention provide an image stitching method for an X-ray imaging system. The X-ray imaging system comprises an X-ray source, and the image stitching method comprises dividing an imaging region of a subject under examination into a first region and a second region, the first region and the second region having an overlapping portion; acquiring a first quantity of X-ray images of the subject under examination by rotating the X-ray source in the first region; acquiring a second quantity of X-ray images of the subject under examination by translating the X-ray source in the second region; and performing image stitching on the first quantity of X-ray images and the second quantity of X-ray images to acquire a medical image of the subject under examination.

The exemplary embodiments of the present invention further provide an X-ray imaging method. The X-ray imaging method comprises determining a first quantity of sub-imaging regions based on a start position and an intermediate position that are set, and determining a second quantity of sub-imaging regions based on the intermediate position and an end position that are set; controlling the X-ray source to move between the start position and the intermediate position, controlling the X-ray source to rotate to respectively align with the first quantity of sub-imaging regions, and controlling the X-ray source to translate to respectively align with the second quantity of sub-imaging regions, so as to acquire a first quantity of X-ray images and a second quantity of X-ray images, respectively; and performing image stitching on the first quantity of X-ray images and the second quantity of X-ray images to acquire a medical image.

The exemplary embodiments of the present invention further provide an X-ray imaging system. The X-ray imaging system comprises an X-ray source capable of emitting X-rays toward a subject under examination, and a control unit capable of being connected to the X-ray source, capable of controlling the movement of the X-ray source, and capable of performing the above-described image stitching method.

The exemplary embodiments of the present invention further provide an X-ray imaging system. The X-ray imaging system comprises an X-ray source capable of emitting X-rays toward a subject under examination, and a control unit capable of being connected to the X-ray source and capable of controlling the movement of the X-ray source. The control unit comprises a region determination unit, a motion control unit, and a stitching unit, wherein the region determination unit is configured to divide an imaging region of the subject under examination into a first region and a second region, the first region and the second region having an overlapping portion; the motion control unit is configured to acquire a first quantity of X-ray images of the subject under examination by rotating the X-ray source in the first region and to acquire a second quantity of X-ray images of the subject under examination by translating the X-ray source in the second region; and the stitching unit is configured to perform image stitching on the first quantity of X-ray images and the second quantity of X-ray images to acquire a medical image of the subject under examination.

Other features and aspects will become apparent from the following detailed description, drawings, and claims.

### BRIEF DESCRIPTION OF THE DRAWINGS

The present invention can be better understood by means of the description of the exemplary embodiments of the present invention in conjunction with the drawings, in which:

FIG. 1 is a schematic diagram of an X-ray imaging system according to some embodiments of the present invention;

FIG. 2 is a schematic diagram of an X-ray imaging system according to some other embodiments of the present invention;

FIG. 3 is a schematic diagram of a control unit according to some embodiments of the present invention;

FIG. 4 is a schematic diagram of determining an imaging region based on a camera image according to some embodiments of the present invention;

FIG. 5 is a schematic diagram of manually determining the imaging region according to some embodiments of the present invention;

FIG. 6 is a schematic diagram of image stitching according to some embodiments of the present invention;

FIG. 7 is a schematic diagram of an image stitching method for an X-ray imaging system according to some embodiments of the present invention; and

FIG. 8 is a flowchart of an X-ray imaging method according to some embodiments of the present invention.

### DETAILED DESCRIPTION

Specific embodiments of the present invention will be described below. It should be noted that in the specific description of these embodiments, for the sake of brevity and conciseness, the present description cannot describe all of the features of the actual embodiments in detail. It should be understood that in the actual implementation process of any implementation, just as in the process of any one engineering project or design project, a variety of specific decisions are often made to achieve specific goals of the developer and to meet system-related or business-related constraints, which may also vary from one implementation to another. Furthermore, it should also be understood that although efforts made in such development processes may be complex and tedious, for those of ordinary skill in the art related to the content disclosed in the present invention, some design, manufacture, or production changes made on the basis of the technical content disclosed in the present disclosure are only common technical means, and should not be construed as the content of the present disclosure being insufficient.

Unless defined otherwise, technical terms or scientific terms used in the claims and description should have the usual meanings that are understood by those of ordinary skill in the technical field to which the present invention belongs. The terms “first” and “second” and similar terms used in the description and claims of the patent application of the present invention do not denote any order, quantity, or importance, but are merely intended to distinguish between different constituents. The terms “one” or “a/an” and similar terms do not express a limitation of quantity, but rather that at least one is present. The terms “include” or “comprise” and similar words indicate that an element or object preceding the terms “include” or “comprise” encompasses elements or objects and equivalent elements thereof listed after the terms “include” or “comprise”, and do not exclude other elements or objects. The terms “connect” or “link” and similar words are not limited to physical or mechanical connections, and are not limited to direct or indirect connections.

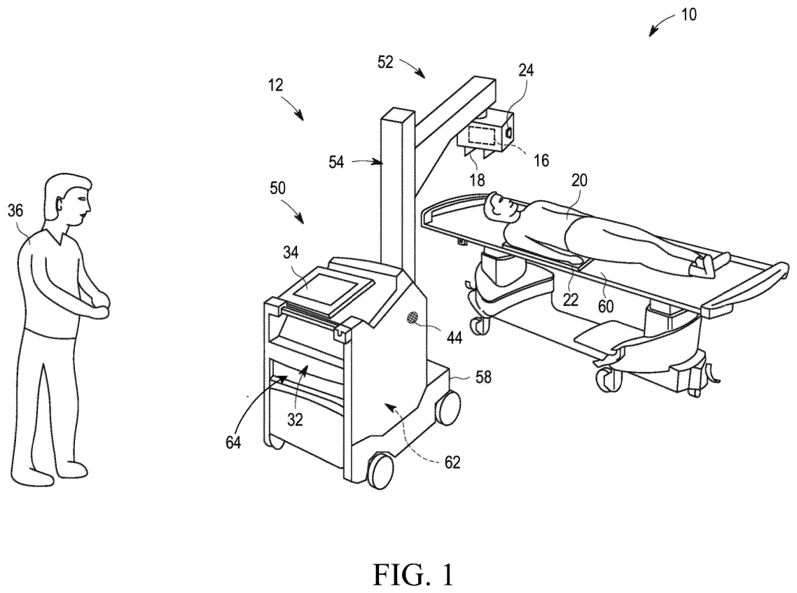

FIG. 1 shows an X-ray imaging system 12 according to some embodiments of the present invention. As shown in FIG. 1, a view 10 for operating an X-ray imaging system includes a movable X-ray imaging system 12. The movable X-ray imaging system can be moved to a patient rehabilitation room, an emergency room, a surgical operating room, or any other space to achieve imaging of a patient 20 without the need to transport the patient 20 to a dedicated (e.g., fixed) X-ray imaging room.

The movable X-ray imaging system 12 includes an X-ray base station 50 and a detector 22. The X-ray base station 50 includes a support arm 52, a support column 54, and a wheeled base 58, where the support arm 52 and the support column 54 are mounted on the wheeled base 58, the wheeled base 58 can drive the entire movable X-ray imaging system 12 to move, the support arm 52 can move vertically along the support column 54 to facilitate positioning an X-ray source 16 and a collimator 18 relative to the patient 20 and the detector 22, and one or both of the support arm 52 and the support column 54 can also be configured to allow the X-ray source 16 to rotate about an axis. The X-ray base station 50 may further include a camera unit 24 to facilitate the positioning of the X-ray source 16 and the collimator 18. Preferably, the camera unit 24 is mounted at a side edge of the X-ray source 16 or the collimator 18. The X-ray base station 50 may further include a speaker 44 to transmit commands audible to the patient.

The patient may be located on a bed 60 (or gurney, table, or any other support) between the X-ray source 16 and the detector 22. During use of an imaging sequence of the movable X-ray imaging system 12, the detector 22 receives X-rays passing through the patient 20 and transmits imaging data to the X-ray base station 50. The detector 22 communicates with the base station 50 by means of a wireless network connection. It is noted that the X-ray imaging system 12 and the detector 22 may use any suitable wireless communication protocol, such as an Institute of Electrical and Electronics Engineers (IEEE) 802.15.4 protocol, an Ultra-Wideband (UWB) communication standard, a Bluetooth communication standard, or any IEEE 802.11 communication standard.

Also, as shown in FIG. 1, the X-ray imaging system 12 includes a workstation 32 and a display 34. More specifically, the X-ray base station 50 has the workstation 32 and the display 34 that enable a user 36 to operate the movable X-ray imaging system 12. The workstation 32 may include buttons, switches, etc., to facilitate operation of the X-ray source 16 and the detector 22, for example. For another example, the workstation 32 may include a visual interface and a device for entering text, such as a keyboard, a touch screen, etc. The X-ray base station 50 further includes a charging apparatus 64 for charging the detector 22 when the detector 22 is not in use.

At least in some embodiments, the X-ray imaging system 12 includes a control unit 62 that can control operation and/or motion, image acquisition, and processing, etc., of the X-ray source 16 and the detector 22.

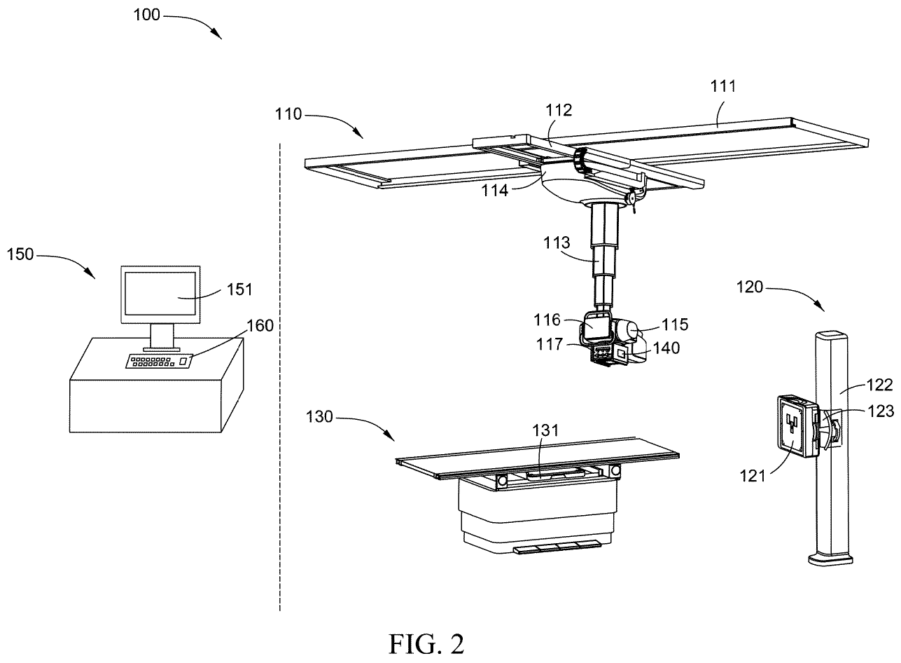

FIG. 2 shows an X-ray imaging system 100 according to some other embodiments of the present invention. As shown in FIG. 2, the X-ray imaging system 100 includes a suspension apparatus 110, a wall stand apparatus 120, and an examination table apparatus 130. The suspension apparatus 110 includes a longitudinal guide rail 111, a transverse guide rail 112, a telescopic cylinder 113, a sliding member 114, a tube assembly 115, and a tube control apparatus 116.

Although the present application is described using a suspended X-ray imaging system shown in FIG. 2 as an example, an image stitching method and apparatus in the present application may also be applied to a ground rail-type X-ray imaging system and a movable X-ray imaging system. Specifically, an X-ray source may be mounted on a ground rail-type cross arm, that is, a cross arm on which the X-ray source is mounted is mounted in a rail on the ground by means of a wall stand, and the X-ray source can carry out motion along the rail, the wall stand, and the cross arm. Certainly, the X-ray source may alternatively be mounted on a mobile cart by means of a telescopic arm.

For case of description, in the present application, the x-axis, y-axis, and z-axis are defined as the x-axis and y-axis being located in the horizontal plane and perpendicular to one another, and the z-axis being perpendicular to the horizontal plane. Specifically, the direction in which the longitudinal guide rail 111 is located is defined as the x-axis, the direction in which the transverse guide rail 112 is located is defined as the y-axis direction, and the direction of extension of the telescopic cylinder 113 is defined as the z-axis direction, and the z-axis direction is the vertical direction.

The longitudinal guide rail 111 and the transverse guide rail 112 are perpendicularly arranged, the longitudinal guide rail 111 being mounted on a ceiling and the transverse guide rail 112 being mounted on the longitudinal guide rail 111. The telescopic cylinder 113 is configured to carry the tube assembly 115.

The sliding member 114 is provided between the transverse guide rail 112 and the telescopic cylinder 113. The sliding member 114 may include components such as a rotating shaft, a motor, and a reel. The motor can drive the reel to rotate around the rotating shaft, which in turn drives the telescopic cylinder 113 to move along the z-axis and/or slide relative to the transverse guide rail. The sliding member 114 is capable of sliding relative to the transverse guide rail 112, i.e., the sliding member 114 is capable of driving the telescopic cylinder 113 and/or the tube assembly 115 to move in the y-axis direction. Further, the transverse guide rail 112 can slide relative to the longitudinal guide rail 111, which in turn drives the telescopic cylinder 113 and/or the tube assembly 115 to move in the x-axis direction.

The telescopic cylinder 113 includes a plurality of cylinders having different inner diameters, and the plurality of cylinders can be sleeved, sequentially from bottom to top, in the cylinder located thereabove, thereby achieving telescoping, and the telescopic cylinder 113 can be telescopic (or movable) in the vertical direction, i.e., the telescopic cylinder 113 can drive the tube assembly 115 to move along the z-axis direction. The lower end of the telescopic cylinder 113 is further provided with a rotating part, and the rotating part can drive the tube assembly 115 to rotate.

Specifically, the X-ray source and a collimator 117 are provided within the tube assembly 115 and the collimator 117 is typically mounted below the X-ray source.

The collimator 117 includes four movable collimator shutters, the four collimator shutters being a material capable of absorbing X-rays, and the four collimator shutters together enclose to form a square or rectangle, and, after enclosing, the four collimator shutters also form an opening in the middle. The opening is the collimator opening, and the size of the collimator 117 opening determines the X-ray irradiation range, i.e., the size of the exposure field of view (FOV). X-rays can pass through the opening of the collimator to a region of interest (ROI) of a subject under examination, and other X-rays are absorbed by the shutters to prevent the subject under examination from absorbing an excess unnecessary dose.

In some embodiments, the X-ray imaging system 100 further includes a camera unit 140, and the camera unit 140 is aligned with the detector so as to be configured to acquire a real-time camera image of the subject under examination. In addition, the camera is able to acquire an image of the detector, etc. Specifically, the camera unit 140 is mounted on the suspension apparatus 110, and further, on the side of the collimator 117.

The camera unit 24/140 may include one or more cameras, for example, a digital camera, an analog camera, etc., or a depth camera, an infrared camera, or an ultraviolet camera, etc., or a 3D camera, a 3D scanner, etc., or a red, green, and blue (RGB) sensor, an RGB depth (RGB-D) sensor, or other devices that can capture color image data of a target subject. In some embodiments, the camera unit 24/140 is further provided with a control module that can control the rotation of the camera unit to adjust the capture range of the camera unit. In other embodiments, the camera unit is a panoramic camera that can take an image of the entire body of the subject under examination.

The camera unit 24/140 can acquire depth information or a depth image of the subject under examination. Typically, the depth information is calculated from a 3D point cloud that is acquired by the camera. In addition, a real-time optical image can be used to acquire at least one of the thickness, height, position, body position, pose, etc., of the subject under examination.

In some embodiments, the camera unit 24/140 may also be a camera unit that is mounted in a fixed position, or fixed in any other way in a scan room. In some embodiments, the optical image acquired by the camera unit 24/140 is not limited to a single optical image, but may also include a dynamic real-time video stream, i.e., a series of real-time optical images.

The tube control apparatus (console) 116 is mounted on the tube assembly 115. The tube control apparatus 116 includes user interfaces such as a display screen and a control button so as to be configured to perform pre-capturing preparations, such as patient selection, protocol selection, positioning, etc.

The movement of the suspension apparatus 110 includes the movement of the tube assembly along the x-axis, y-axis, and z-axis, as well as the rotation of the tube assembly in the horizontal plane (the axis of rotation is parallel to or overlaps with the z-axis) and in the vertical plane (the axis of rotation is parallel to the y-axis). In the above motion, a motor is usually used to drive a rotating shaft which in turn drives corresponding components to rotate in order to achieve the corresponding movement or rotation, and the corresponding control components are generally mounted in the sliding member 114. The X-ray imaging system further includes a motion control unit (not shown in the figures) that is capable of controlling the movement of the suspension apparatus 110, and furthermore, the motion control unit is capable of receiving a control signal to control the corresponding component to move accordingly to drive the arena assembly to reach a preset or specified position.

The wall stand apparatus 120 includes a first detector 121, a wall stand 122, and a connecting member 123. The connecting member 123 includes a support arm that is vertically connected in the height direction of the wall stand 122 and a rotating bracket that is mounted on the support arm, and the first detector 121 is mounted on the rotating bracket. The wall stand apparatus 120 further includes a detector driving apparatus that is arranged between the rotating bracket and the first detector 121, which is driven by the detector driving apparatus to move in a direction parallel to the height direction of the wall stand 122 in the plane held by the rotating bracket, and the first detector 121 can further be rotated relative to the support arm to form an angle with the wall stand. The first detector 121 has a plate-like structure of which the orientation is variable so that an X-ray incident surface can become vertical or horizontal depending on the incident direction of the X-rays.

A second detector 131 is included on the examination table apparatus 130, and the selection or use of the first detector 121 and the second detector 131 may be determined based on a capture site of a patient and/or a capture protocol, or may be determined based on the position of the subject under examination that is obtained from a camera capture, so as to conduct a supine, prone or standing capture examination. FIG. 2 only shows an example diagram of a wall stand and an examination table, and it should be understood by those skilled in the art that wall stands and/or examination tables of any form or arrangement can be selected, or only the wall stand can be mounted, and the wall stand and/or examination table is not intended to limit the overall solution of the present application.

The X-ray imaging system 100 further includes a display unit 150 that is operably connected to the camera unit and includes a user interface 151 configured to display the real-time optical image, the X-ray images, the medical image, the information of the subject under examination, an exposure parameter setting interface, an image post-processing interface, etc.

Specifically, the display unit 150 can include any form of display screen, which may be a main display screen that is located in the control room, a display screen of the tube control apparatus 116 that is located in the scan room, or a mobile display, such as a tablet, a cell phone, etc.

The X-ray imaging system further includes an input unit 160, configured to receive a user operation. The input unit 160 can include an input device such as a touchscreen, a keyboard, a mouse, a voice-activated control unit, or any other suitable input device, and a user can input an operation signal/control signal into the control unit by means of the input unit 160.

The X-ray imaging system 100 further includes a control unit (not shown in the figures), which may be a main control unit that is located in the control room, a tube control unit that is mounted on the suspension apparatus, a mobile or portable control unit, or any combination of the above. The control unit may include a source control unit and a detector control unit. The source control unit is configured to command the X-ray source to emit X-rays for image exposure. The detector control unit is configured to select an appropriate detector from among a plurality of detectors and to coordinate the control of various detector functions, such as automatically selecting a corresponding detector according to the position or pose of the subject under examination, or may perform various signal processing and filtering functions, and is specifically used for the initial adjustment of the dynamic range, interleaving of digital image data, etc. In some embodiments, the control unit may provide power and timing signals for controlling the operation of the X-ray source and the detector.

In some embodiments, the control unit may also be configured to use a digitized signal to reconstruct one or more required images and/or determine useful diagnostic information corresponding to the patient, wherein the control unit may include one or more dedicated processors, graphics processing units, digital signal processors, microcomputers, microcontrollers, application-specific integrated circuits (ASICs), field-programmable gate arrays (FPGAs), or other appropriate processing apparatuses.

Of course, the X-ray imaging system may also include other numbers or configurations or forms of control units, for example, the control unit may be local (e.g., co-located with one or more X-ray imaging systems 100, e.g., within the same facility and/or the same local network). In other implementations, the control unit may be remote and thus only accessible by means of a remote connection (for example, by means of the Internet or other available remote access technologies). In a specific implementation, the control unit may also be configured in a manner similar to the configuration of cloud technology, and may be accessed and/or used in a manner substantially similar to the manner of accessing and using other cloud-based systems.

In one embodiment, the X-ray imaging system 100 further includes an operator workstation, the operator workstation allowing the user to receive and evaluate the reconstructed image, and input a control instruction (an operation signal or a control signal). The operator workstation may include a user interface (or user input device) in a certain form of operator interface, such as a keyboard, a mouse, a voice activated control apparatus, or any other suitable input device, such that an operator may input an operation signal/control signal to the control apparatus by means of the user interface.

Generally, the size of an obtained medical image is generally equal to the size of the X-ray detector (the exposure field of view). If the region of interest is within the size of the X-ray detector (the exposure field of view), then the entire region of interest can be completely presented in one image. If the region of interest exceeds the size of the X-ray detector (the exposure field of view), then the entire region of interest cannot be completely presented in one image, and it is necessary to divide the region of interest into a plurality of sub-imaging regions, respectively perform exposure imaging on each sub-imaging region, and stitch a plurality of acquired X-ray images together to acquire a complete medical image.

Specifically, in the present application, a region of interest of the subject under examination is defined as an imaging region, that is, an entire exposure region, a region determined according to the size of the detector or a collimation region is defined as a sub-imaging region, an image acquired by each sub-imaging region is defined as an X-ray image, and an image of the entire imaging region obtained by stitching the X-ray images is defined as a medical image.

In current clinical practice, it is sometimes necessary to acquire a whole-body image of the subject under examination to determine an overall relationship or association among various parts of the subject under examination, for example, it is necessary to determine or view the condition of the lower limbs from the state of the spine, or determine the influence of the lower limbs on the spine from the condition of the lower limbs, etc.

In general, there are currently two commonly used image stitching modes. One is performing image stitching by rotating an X-ray source to align with a plurality of regions and then implement imaging of the plurality of regions, and the other one is to implement imaging and image stitching by translating an X-ray source to align with a plurality of regions. The two modes have respective advantages and disadvantages, where for an imaging region of the same size, the means in which the X-ray source is rotated requires a small quantity of images, that is, the quantity of divided sub-imaging regions is small, and the quantity of obtained X-ray images is small, so the time required for imaging is also fast, there is also less tissue deformation in overlapping regions between a plurality of adjacent sub-imaging regions, and accordingly, the accuracy of registration is high. For the means in which the X-ray source is translated, the overall tissue deformation of a medical image that is completely stitched is small, and accurate and precise measurement can be performed on the overall medical image, but its disadvantage corresponds to the advantages of the mode implemented by rotating the X-ray source, that is, the quantity of divided sub-imaging regions is higher, the imaging time is longer, etc.

For whole-body imaging, the applicant has found that if a single stitching mode is used, then an obtained whole-body image will be inaccurate and accordingly will not provide an accurate reference for the diagnosis of a doctor or user, for example, in the whole-body image, the doctor wants to see the precise shape of the spine, while for the lower limbs, the doctor wants to measure the length of each part or the whole of the lower limbs, and therefore, the whole-body image obtained by the use of the single stitching mode does not satisfy the doctor's requirements. Therefore, the applicant proposes a combined image stitching mode. For an imaging region where the shape of the spine needs to be completely restored, imaging is performed by means of rotating an X-ray source, and for an imaging region where the length of the lower limbs needs to be restored, imaging is performed by means of translating the X-ray source. By combining the two stitching manners, the advantages of the two stitching modes can be made compatible, and the imaging needs of the doctor for different regions can be satisfied.

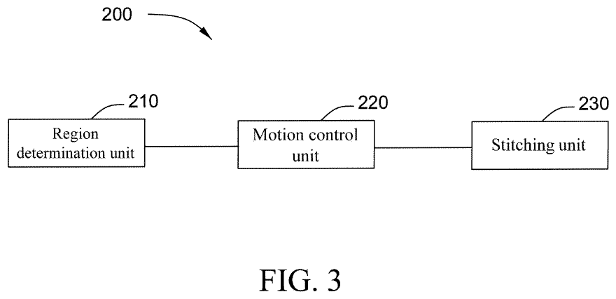

FIG. 3 shows a schematic diagram of a control unit 200 according to some embodiments of the present application. As shown in FIG. 3, the control unit 200 in the present application includes a region determination unit 210, a motion control unit 220, and a stitching unit 230. Specifically, the region determination unit 210 is configured to divide an imaging region of a subject under examination into a first region and a second region, where the first region and the second region have an overlapping portion. The motion control unit 220 can control the movement of the X-ray source, acquire a first quantity of X-ray images of the subject under examination by rotating the X-ray source in the first region, and acquire a second quantity of X-ray images of the subject under examination by translating the X-ray source in the second region. The stitching unit 230 can perform image stitching on the first quantity of X-ray images and the second quantity of X-ray images to acquire a medical image of the subject under examination.

In some embodiments, the region determination unit 210 can further be configured to determine a start position, an intermediate position, and an end position of the subject under examination for image stitching to determine the first region and the second region of the subject under examination.

In some embodiments, a region between the start position and the intermediate position may be defined as one of the first region and the second region, and a region between a temporary position and the end position constitutes the other of the first region and the second region, the temporary position being a position adjacent to the start position and having a preset distance from the intermediate position, where the overlapping portion of the first region and the second region is between the temporary position and the intermediate position.

Specifically, the region between the start position and the intermediate position may be defined as the first region and the region between the temporary position and the end position may be defined as the second region, or vice versa, the region between the temporary position and the end position may be defined as the first region and the region between the start position and the intermediate position may be defined as the second region.

Specifically, in order to achieve final whole-body stitching, a preset overlapping region needs to be respectively provided between sub-imaging regions within each region and between the regions, e.g., an overlapping region of 7 cm, and therefore, a portion between the temporary position and the intermediate position is the overlapping region of the first region and the second region.

Specifically, for whole-body imaging, the intermediate position may be a position where the pelvis is located. Preferably, the intermediate position is a position of a lower edge of where the pelvis is located, and the temporary position is a position of an upper edge of where the pelvis is located, that is, the position where the pelvis is located is defined as the overlapping region of the first region and the second region. The reason why the position where the pelvis is located is defined as the overlapping region is that, (I), the position where the pelvis is located is easily recognized and the two regions can be easily divided, and (II), a stitched image obtained from the overlapping region will have a large deformation, and the shape or size of the pelvis has substantially no influence on diagnostic aids.

Specifically, the start position, the intermediate position, and the end position of the subject under examination may be manually determined by a user, or determined based on automatic recognition of an anatomical site or keypoint, and of course, may also be automatically or manually determined based on assistance of a camera image.

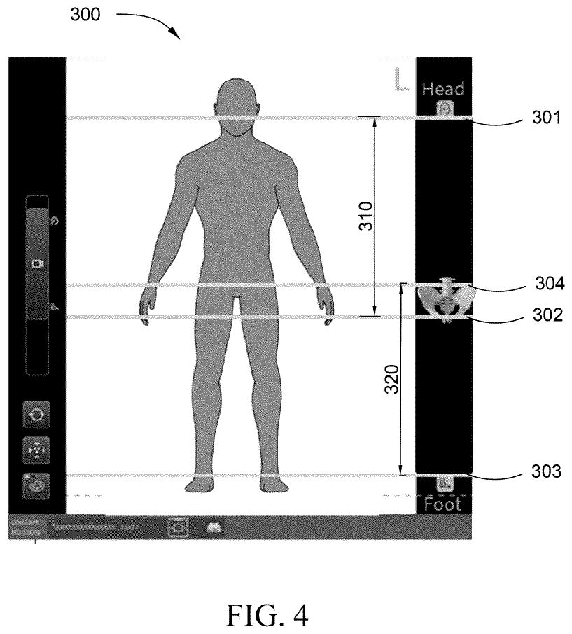

In some embodiments, FIG. 4 shows a schematic diagram of determining an imaging region based on a camera image according to some embodiments of the present invention. As shown in FIG. 4, a camera image 300 acquired by a camera unit mounted at a side edge of a suspension apparatus or an X-ray source or in a scanning room can be displayed on a user interface of a display unit, and of course, other exposure parameters or adjustment buttons or the like can also be displayed on the user interface in addition to the camera image 300.

Specifically, a start position 301, an intermediate position 302, and an end position 303 (hereinafter collectively referred to as boundary positions) of an imaging region may also be displayed in a superimposed manner on the camera image 300, which may be a still image or a one-frame image in a video stream acquired in real time. For example, the start position 301, the intermediate position 302, and the end position 303 may be indicated on the camera image by using indication lines such as a solid line or a dotted line, the indication line may be the same as, smaller than, or larger than the width of the camera image, and the shape, position, or size of the indication line are not limited in the present application.

In some embodiments, initial positions of the start position 301, the intermediate position 302, and the end position 303 may be initial recommended positions calculated or acquired by a control unit based on basic information such as an exposure site, and the age or a disease of a subject under examination, or may be initial positions determined by the control unit based on identified information of an anatomical site or a keypoint, or may be manually determined by a user on the camera image. The initial position may be directly used as a boundary position of an exposure region. Of course, based on the initial position, the user may also perform position adjustment by using an adjusting tool provided on the user interface, and the adjusting tool may be an adjusting button provided around the indication line at each position, or an adjusting tool provided in an operation region on the outer side of the camera image, or the like. Of course, the user may directly pull or drag the indication line to move the same. How to adjust the positions of the start position 301, the intermediate position 302, and the end position 303 is not limited in the present application, as long as the user can adjust a position where any boundary (the start position 301, the intermediate position 302, and the end position 303) is located.

In some embodiments, the start position is generally a position closer to the head of the subject under examination, the end position is a position closer to the feet of the subject under examination, and the intermediate position is a position located between the start position and the end position.

In some embodiments, in addition to the indication lines indicating the start position 301, the intermediate position 302, and the end position 303, the name of each position (e.g., head position, intermediate position, foot position) and/or the name of the anatomical site (e.g., head, pelvis, foot) corresponding to each position and/or the thumbnail of the anatomical site corresponding to each position, etc., may be displayed within the camera image or the operation region around the camera image. Of course, such display may not be performed, or other content may be displayed.

In some embodiments, a temporary position 304 may also be displayed in a superimposed manner in the camera image, and the temporary position is determined by means of a preset distance from the intermediate position, or may be determined by identifying an upper edge of a position where the pelvis is located. Of course, an indication line of the temporary position may not be shown in the camera image.

Specifically, after the start position 301, the intermediate position 302, and the end position 303 are confirmed, a first region 310 is located between the start position 301 and the intermediate position 302, and a second region 320 is located between the temporary position 304 (having the preset distance from the intermediate position 302) and the end position 303.

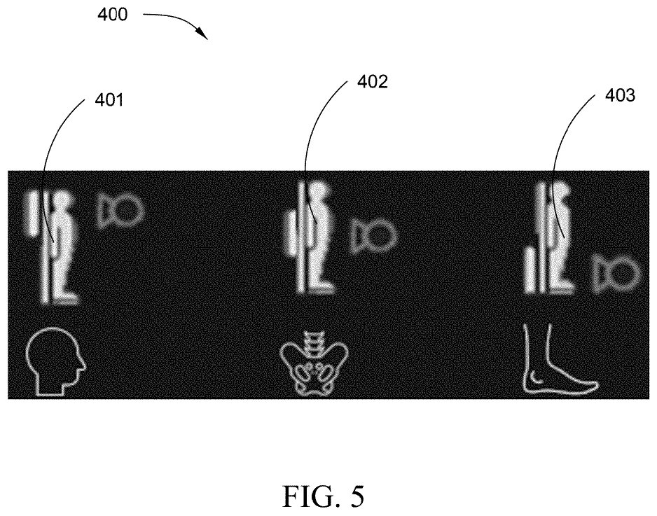

In some embodiments, FIG. 5 shows a schematic diagram of manually determining the imaging region according to some embodiments of the present invention. As shown in FIG. 5, a scanning protocol or exposure protocol for whole-body imaging is set in the X-ray imaging system. When a scan site of the subject under examination includes whole-body imaging or when the user selects, by means of a user interface, the protocol for whole-body imaging, the user interface on a display unit displays a prompt operation interface for confirming a boundary position as shown in FIG. 5. Of course, the interface shown in FIG. 5 may be displayed on the display unit as the entire user interface or may be displayed in the display unit through a part of the user interface.

In the process of confirming the boundary positions, the suspension apparatus can be automatically or manually controlled to move along the head to the feet of the subject under examination. When the user confirms that a current position is the start position, the user can press a control button, the position of the suspension apparatus at this time is recorded as an initial position, then the suspension apparatus continues to move. When the user presses the control button again, the position where the suspension apparatus is aligned at this time is recorded as the intermediate position, and similarly, the suspension apparatus continues to move. When the user presses the control button again, the position where the suspension apparatus is aligned at this time is recorded as the end position. Specifically, the suspension apparatus may be manually controlled to move by the user, or may be set according to the scanning protocol for whole-body imaging, for example, the suspension apparatus moves along the subject under examination at a preset slow speed without emitting any X-rays, etc.

Furthermore, during movement of the suspension apparatus, a laser lamp or other apparatus (e.g., a projection apparatus or other light emitting apparatus mounted on the collimator) that can emit a reference position built in the collimator can emit a position reference line (e.g., a laser line) that can indicate a current position on the body of the subject under examination, and the user can determine whether the current position is a start point, an intermediate point, and an end point of an imaging region or an exposure region based on the position reference line to confirm the start position, the intermediate position, and the end position of the region.

In some embodiments, the user can confirm corresponding boundary positions through different positions in the prompt operation interface (e.g., user interface 400) as shown in FIG. 5. Specifically, when the position where the suspension apparatus is currently aligned is determined as the start position, the user can click on a start position icon 401 to determine and store the position of the suspension apparatus, and of course, the user can also click on an intermediate position icon 402 and an end position icon 403 to determine and store the position of the suspension apparatus. In some preferred embodiments, the confirmation of the boundary positions needs to be performed in order of the start position to the intermediate position to the end position. In some preferred embodiments, in the user interface 400, the position icons 401, 402, and 403 can be respectively highlighted or enlarged or otherwise processed to prompt which specific boundary position the user currently needs to confirm. Of course, in addition to position confirmation in the graphical user interface, the boundary positions may be confirmed in any other suitable manner, for example, a hand-held positioning apparatus, etc. Of course, the position icons or the user interface are not limited to those shown in FIG. 5, and there can be other arrangement manners or icon types, for example, the position icons may be arranged vertically, or the position icons may be shown in text or other graphics.

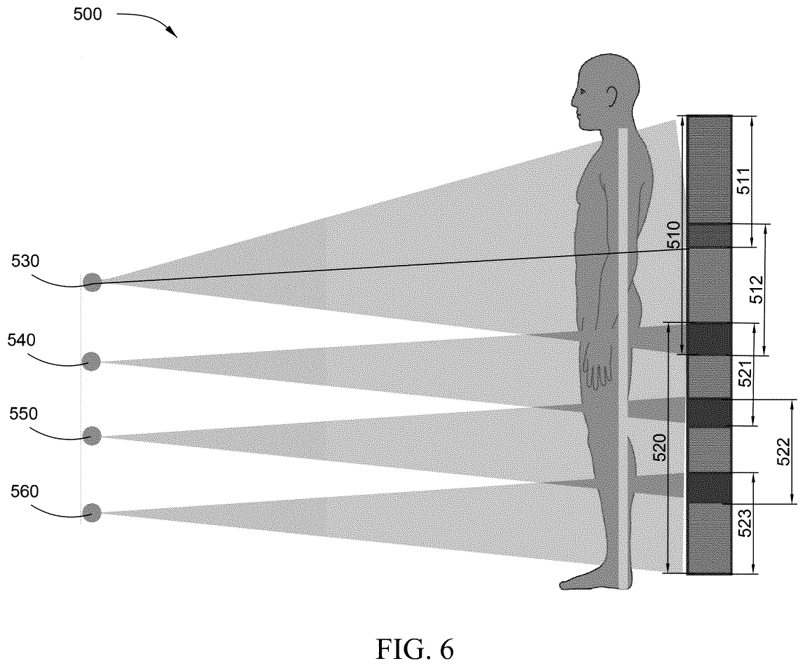

When the boundary positions (the start position, the intermediate position, and the end position) of the imaging region are confirmed by the region determination unit 210, the region between the start position and the intermediate position is defined as one of the first region and the second region, and the region between the temporary position having a preset distance above the intermediate position and the end position is defined as the other of the first region and the second region. FIG. 6 shows a schematic diagram of image stitching according to some embodiments of the present application. As shown in FIG. 6, the region between the start position and the intermediate position is referred to as a first region 510, and the region between the temporary position having a preset distance above the intermediate position and the end position is referred to as a second region 520.

Further, the region determination unit 210 can further divide each region into a different quantity of sub-imaging regions based on one or more of the definitions of the size of the detector, the size of the collimation region, the width and/or thickness of the subject under examination, the type of image stitching, or the size of the overlapping region. Specifically, assuming that the imaging region corresponding to the first region is the spine, the imaging region corresponding to the second region is the lower limbs (including the pelvis, the legs, and the ankles), that is, the image stitching type corresponding to the first region is the stitching mode implemented by rotating an X-ray source, the region determination unit 210 can divide the first region into a first quantity of sub-imaging regions (e.g., including a first sub-imaging region 511 and a second sub-imaging region 512) according to the size of the detector and the size of the overlapping region. Since the image stitching type corresponding to the second region is a stitching mode implemented by translating the X-ray source, the region determination unit 210 can divide the second region into a second quantity of sub-imaging regions (e.g., including a third sub-imaging region 521, a fourth sub-imaging region 522, and a fifth sub-imaging region 523) according to the size of the detector and the size of the overlapping region. Of course, the first region and the second region can be divided into different quantities of sub-imaging regions based on factors such as the height of the subject under examination, the head-to-pelvis length, and the length of the lower limbs. For example, for a child, it is possible that only one image needs to be taken from the head to the pelvis. Each of the first quantity and the second quantity is an integer greater than or equal to 1.

After the region determination unit 210 determines the sub-imaging regions, the motion control unit 220 can control the X-ray source to rotate and/or translate to align with each sub-imaging region separately and simultaneously acquire an X-ray image corresponding to each sub-imaging region.

Specifically, the motion control unit 220 can control the X-ray source to reach an initial position 530 and an initial angle, where the initial position 530 substantially corresponds to a central position of the first region 510, and the initial angle refers to X-rays emitted from the X-ray source being substantially horizontal (when the subject under examination stands in front of the wall stand) or vertical (when the subject under examination lies on the examination table), i.e., perpendicular to a body surface of the subject under examination, then the X-ray source is rotated to align with the first quantity of sub-imaging regions in the first region 510 to acquire a first quantity of X-ray images of the subject under examination, and next, the X-ray source can be rotated back to the initial angle and moved to a central position of each sub-imaging region in the second region 520 to respectively align with each sub-imaging region to acquire a second quantity of X-ray images of the subject under examination.

Specifically, after the X-ray source is located at the initial position 530, the X-ray source is rotated to a first angle that aligns with a first sub-imaging region 511, so that the X-ray source can align with the first sub-imaging region 511 to emit X-rays and acquire a first X-ray image. The X-ray source is then rotated to a second angle that aligns with a second sub-imaging region 512, so that the X-ray source can align with the second sub-imaging region 512 to emit X-rays and acquire a second X-ray image.

Next, the X-ray source can be rotated back to the initial angle and moved to a second position 540 to align with a third sub-imaging region 521 so that the X-ray source can align with the third sub-imaging region 521 to emit X-rays and acquire a third X-ray image, then moved to a third position 550 to align with a fourth sub-imaging region 522 and acquire a fourth X-ray image, and then moved to a fourth position 560 to align with a fifth sub-imaging region 523 and acquire a fifth X-ray image. The second position, the third position, and the fourth position substantially align with the centers of the third sub-imaging region, the fourth sub-imaging region, and the fifth sub-imaging region, respectively.

After the first quantity of X-ray images and the second quantity of X-ray images are acquired, the stitching unit 230 can further perform image stitching on the first quantity of X-ray images and the second quantity of X-ray images to acquire a medical image of the subject under examination.

In some embodiments, the stitching unit 230 can perform image stitching on the first quantity of X-ray images first to acquire a first region image, then perform image stitching on the second quantity of X-ray images to acquire a second region image, and finally perform stitching on the first and second region images to acquire a final panoramic or whole-body image. Specifically, the stitching unit 230 can stitch the X-ray images acquired in the first region first, then stitch the X-ray images acquired in the second region, and then stitch the images of the two regions together to acquire the final whole-body image.

In other embodiments, the stitching unit 230 can perform stitching one at a time in the order of acquiring the X-ray images, and stitch an (m+2)th image acquired from the first quantity of X-ray images or the second quantity of X-ray images and an (m) th temporary image to acquire the medical image, where m is an integer greater than or equal to 1, and the (m)th temporary image refers to an intermediate image obtained by stitching the first (m+1) images. Specifically, a first X-ray image and a second X-ray image are stitched first to obtain a first temporary image, then an acquired third X-ray image and the first temporary image are stitched to obtain a second temporary image, then an acquired fourth X-ray image and the second temporary image are stitched to obtain a third temporary image, and such a process is repeated until all the X-ray images are completely stitched to obtain the final whole-body image.

Specifically, the stitching unit 230 can stitch any two images in any suitable manner, for example, same feature points in the two images may be identified and aligned, which is not limited in the present application.

In some embodiments, the stitching unit 230 can further perform processing, e.g., de-noising, de-artifacting, etc., on the images stitched, to acquire a medical image having a higher image quality. The medical image can be displayed on the user interface of the display unit, and some measuring tools or measuring dimensions, etc., can also be displayed in a superimposed manner on the medical image.

Although the control unit is divided into three unit modules in the above embodiment, it should be understood by those skilled in the art that the three unit modules can also be integrated, that is, the control unit of the X-ray imaging system according to some embodiments of the present application can be configured to: divide an imaging region of a subject under examination into a first region and a second region, the first region and the second region having an overlapping portion; acquire a first quantity of X-ray images of the subject under examination by rotating the X-ray source in the first region; acquire a second quantity of X-ray images of the subject under examination by translating the X-ray source in the second region; and perform image stitching on the first quantity of X-ray images and the second quantity of X-ray images to acquire a medical image of the subject under examination.

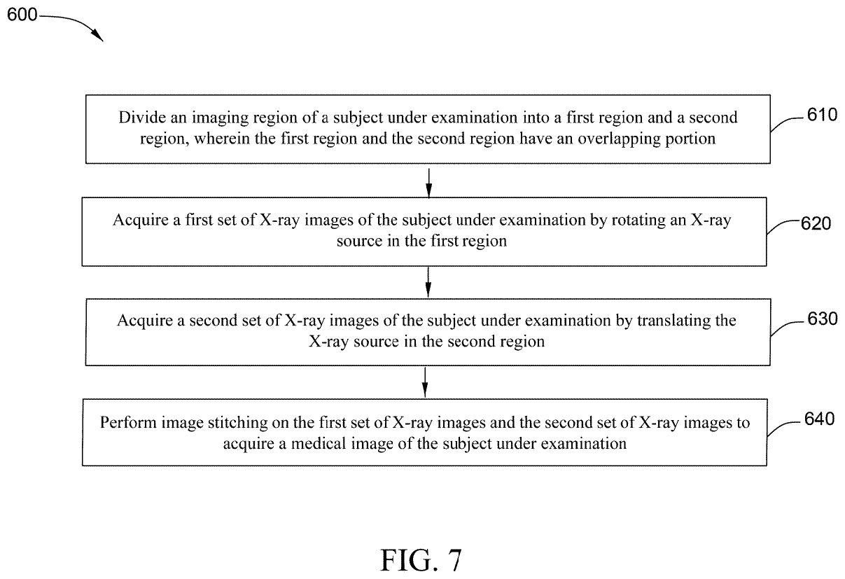

FIG. 7 is a flowchart of an image stitching method for an X-ray imaging system according to some embodiments of the present invention. As shown in FIG. 7, an X-ray imaging method 600 includes step 610, step 620, step 630, and step 640.

In step 610, an imaging region of a subject under examination is divided into a first region and a second region, the first region and the second region having an overlapping portion. Specifically, the first region is a region including the spine of the subject under examination, and the second region is a region including the lower limbs of the subject under examination.

Step 610 further includes determining a start position, an intermediate position, and an end position of the subject under examination for image stitching to determine the first region and the second region of the subject under examination. In some preferred embodiments, the intermediate position is a position where the pelvis is located.

Specifically, a region between the start position and the intermediate position constitutes one of the first region and the second region, a region between a temporary position and the end position constitutes the other of the first region and the second region, the temporary position is a position adjacent to the start position and having a preset distance from the intermediate position, and the overlapping portion is between the temporary position and the intermediate position.

In some embodiments, the determining a start position, an intermediate position, and an end position of the subject under examination for image stitching includes determining the start position, the intermediate position, and the end position based on an acquired camera image. Specifically, the camera image is processed to identify an anatomical site or a keypoint of the subject under examination, and then the start position, the intermediate position, and the end position are determined based on the anatomical site or the keypoint.

In other embodiments, the start position, the intermediate position, and the end position for image stitching can also be determined manually.

In step 620, a first quantity of X-ray images of the subject under examination are acquired by rotating an X-ray source in the first region.

Specifically, the acquiring a first quantity of X-ray images of the subject under examination includes: dividing the first region into a first quantity of sub-imaging regions; controlling the X-ray source to respectively rotate by a first quantity of angles to align with the first quantity of sub-imaging regions, respectively; and acquiring the first quantity of X-ray images corresponding to the first quantity of sub-imaging regions.

In step 630, a second quantity of X-ray images of the subject under examination are acquired by translating the X-ray source in the second region of the subject under examination.

Specifically, the acquiring a second quantity of X-ray images of the subject under examination includes: dividing the second region into a second quantity of sub-imaging regions; controlling the X-ray source to respectively translate to a second quantity of positions to align with the second quantity of sub-imaging regions, respectively; and acquiring the second quantity of X-ray images corresponding to the second quantity of sub-imaging regions.

In step 640, image stitching is performed on the first quantity of X-ray images and the second quantity of X-ray images to acquire a medical image of the subject under examination.

In some embodiments, image stitching can be performed on the first quantity of X-ray images first to acquire a first region image, then image stitching is performed on the second quantity of X-ray images to acquire a second region image, and finally the first and second region images are stitched to acquire a final panoramic or whole-body image. Specifically, the X-ray images acquired in the first region can be stitched first, then the X-ray images acquired in the second region can be stitched, and then the images of the two regions are stitched together to acquire the final whole-body image.

In other embodiments, stitching can be performed one at a time in the order of acquiring the X-ray images, and an (m+2)th image acquired from the first quantity of X-ray images or the second quantity of X-ray images and an (m)th temporary image are stitched to acquire the medical image, where m is an integer greater than or equal to 1, and the (m) th temporary image refers to an intermediate image obtained by stitching the first (m+1) images. Specifically, a first X-ray image and a second X-ray image are stitched first to obtain a first temporary image, then an acquired third X-ray image and the first temporary image are stitched to obtain a second temporary image, then an acquired fourth X-ray image and the second temporary image are stitched to obtain a third temporary image, and such a process is repeated until all the X-ray images are completely stitched to obtain the final whole-body image.

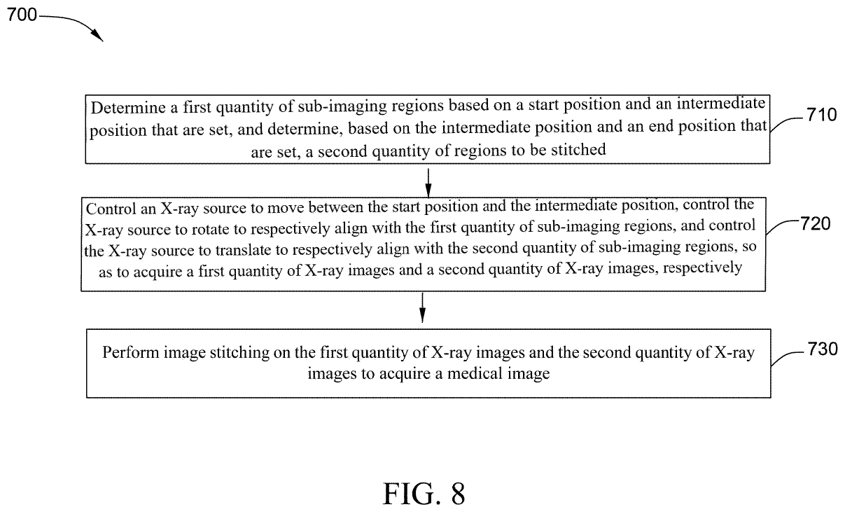

FIG. 8 is a flowchart of an X-ray imaging method according to some embodiments of the present invention. As shown in FIG. 8, the X-ray imaging method 700 includes step 710, step 720, and step 730.

In step 710, a first quantity of sub-imaging regions are determined based on a start position and an intermediate position that are set, and a second quantity of sub-imaging regions are determined based on the intermediate position and an end position that are set.

In step 720, an X-ray source is controlled to move between the start position and the intermediate position, the X-ray source is controlled to rotate to respectively align with the first quantity of sub-imaging regions, and the X-ray source is controlled to translate to respectively align with the second quantity of sub-imaging regions, so as to acquire a first quantity of X-ray images and a second quantity of X-ray images, respectively.

In step 730, image stitching is performed on the first quantity of X-ray images and the second quantity of X-ray images to acquire a medical image.

In an image stitching method according to some embodiments of the present invention, first, according to the present application, by setting automatic acquisition of a whole-body image of a subject under examination, a user can see the whole-body image through a film to analyze, for example, the influence of the spine on the lower limbs or the influence of the lower limbs on the spine, etc., thereby providing accurate assistance for a physician's diagnosis. Furthermore, by imaging the subject under examination respectively through a combination of two image stitching manners, i.e., rotating the X-ray source and translating the X-ray source, the advantages of the two image stitching manners can be combined, so that the acquired shape of the spine is more precise, and the acquired measurement size of the lower limbs can be precise, thereby meeting the needs of the user.

Exemplary embodiments of the present invention provide an image stitching method for an X-ray imaging system. The X-ray imaging system includes an X-ray source, and the image stitching method includes dividing an imaging region of a subject under examination into a first region and a second region, the first region and the second region having an overlapping portion; acquiring a first quantity of X-ray images of the subject under examination by rotating the X-ray source in the first region; acquiring a second quantity of X-ray images of the subject under examination by translating the X-ray source in the second region; and performing image stitching on the first quantity of X-ray images and the second quantity of X-ray images to acquire a medical image of the subject under examination.

Specifically, the dividing an imaging region of the subject under examination into a first region and a second region includes determining a start position, an intermediate position, and an end position of the imaging region to determine the first region and the second region.

Specifically, a region between the start position and the intermediate position constitutes one of the first region and the second region, a region between a temporary position and the end position constitutes the other of the first region and the second region, the temporary position is a position adjacent to the start position and having a preset distance from the intermediate position, and the overlapping portion is between the temporary position and the intermediate position.

Specifically, the X-ray imaging system further includes a camera unit configured to acquire a camera image of the subject under examination, and the determining a start position, an intermediate position, and an end position of the imaging region of the subject under examination comprises determining the start position, the intermediate position, and the end position based on the acquired camera image.

Specifically, the determining a start position, an intermediate position, and an end position based on the acquired camera image includes performing image processing on the camera image to identify information of an anatomical site or a keypoint of the subject under examination, and determining the start position, the intermediate position, and the end position based on the information of the anatomical site or the keypoint.

Specifically, the intermediate position is a position where the pelvis is located.

Specifically, the first region is a region including the spine of the subject under examination, and the second region is a region including the lower limbs of the subject under examination.

Specifically, the acquiring a first quantity of X-ray images of the subject under examination includes dividing the first region into a first quantity of sub-imaging regions, controlling the X-ray source to respectively rotate by a first quantity of angles to align with the first quantity of sub-imaging regions, respectively, and acquiring the first quantity of X-ray images corresponding to the first quantity of sub-imaging regions.

Specifically, the acquiring a second quantity of X-ray images of the subject under examination includes dividing the second region into a second quantity of sub-imaging regions, controlling the X-ray source to respectively translate to a second quantity of positions to align with the second quantity of sub-imaging regions, respectively, and acquiring the second quantity of X-ray images corresponding to the second quantity of sub-imaging regions.

Specifically, the performing image stitching on the first quantity of X-ray images and the second quantity of X-ray images includes performing image stitching on the first quantity of X-ray images to acquire a first region image; performing image stitching on the second quantity of X-ray images to acquire a second region image; and stitching the first region image and the second region image to acquire the medical image; alternatively, stitching an (m+2)th image acquired from the first quantity of X-ray images or the second quantity of X-ray images and an (m)th temporary image to acquire the medical image, wherein m is an integer greater than or equal to 1, and the (m)th temporary image refers to an intermediate image obtained by stitching the first (m+1) images.

The exemplary embodiments of the present invention further provide an X-ray imaging method. The X-ray imaging method includes determining a first quantity of sub-imaging regions based on a start position and an intermediate position that are set, and determining a second quantity of sub-imaging regions based on the intermediate position and an end position that are set; controlling an X-ray source to move between the start position and the intermediate position, controlling the X-ray source to rotate to respectively align with the first quantity of sub-imaging regions, and controlling the X-ray source to translate to respectively align with the second quantity of sub-imaging regions, so as to acquire a first quantity of X-ray images and a second quantity of X-ray images, respectively; and performing image stitching on the first quantity of X-ray images and the second quantity of X-ray images to acquire a medical image.

The exemplary embodiments of the present invention further provide an X-ray imaging system. The X-ray imaging system includes an X-ray source that can emit X-rays toward a subject under examination, and a control unit that can be connected to the X-ray source, can control the movement of the X-ray source, and can perform the image stitching method or the X-ray imaging method described above.

The exemplary embodiments of the present invention further provide an X-ray imaging system. The X-ray imaging system includes an X-ray source that can emit X-rays toward a subject under examination, and a control unit that can be connected to the X-ray source and can control the movement of the X-ray source. The control unit includes a region determination unit, a motion control unit, and a stitching unit, where the region determination unit is configured to divide an imaging region of the subject under examination into a first region and a second region, the first region and the second region having an overlapping portion; the motion control unit is configured to acquire a first quantity of X-ray images of the subject under examination by rotating the X-ray source in the first region and to acquire a second quantity of X-ray images of the subject under examination by translating the X-ray source in the second region; and the stitching unit is configured to perform image stitching on the first quantity of X-ray images and the second quantity of X-ray images to acquire a medical image of the subject under examination.

The present invention may further provide a non-transitory computer-readable storage medium for storing an instruction set and/or a computer program. When executed by a computer, the instruction set and/or computer program causes the computer to perform the image processing distribution method. The computer executing the instruction set and/or computer program may be a computer of a medical imaging system, or may be other apparatuses/modules of the medical imaging system. In one embodiment, the instruction set and/or computer program may be programmed into a processor/control apparatus of the computer.

Specifically, when executed by the computer, the instruction set and/or computer program causes the computer to:
divide an imaging region of a subject under examination into a first region and a second region, the first region and the second region having an overlapping portion;
acquire a first quantity of X-ray images of the subject under examination by rotating the X-ray source in the first region;
acquire a second quantity of X-ray images of the subject under examination by translating the X-ray source in the second region; and
perform image stitching on the first quantity of X-ray images and the second quantity of X-ray images to acquire a medical image of the subject under examination; or
determine a first quantity of sub-imaging regions based on a start position and an intermediate position that are set, and determine a second quantity of sub-imaging regions based on the intermediate position and an end position that are set;
control an X-ray source to move between the start position and the intermediate position, control the X-ray source to rotate to respectively align with the first quantity of sub-imaging regions, and control the X-ray source to translate to respectively align with the second quantity of sub-imaging regions, so as to acquire a first quantity of X-ray images and a second quantity of X-ray images, respectively; and
perform image stitching on the first quantity of X-ray images and the second quantity of X-ray images to acquire a medical image.

The instructions described above may be combined into one instruction for execution, and any of the instructions may also be split into a plurality of instructions for execution. Moreover, the present invention is not limited to the instruction execution order described above.

As used herein, the term “computer” may include any processor-based or microprocessor-based system, including a system using a microcontrol apparatus, a reduced instruction set computer (RISC), an application-specific integrated circuit (ASIC), a logic circuit, and any other circuit or processor capable of performing the functions described herein. The examples above are exemplary only and are not intended to limit the definition and/or meaning of the term “computer” in any way.

Some exemplary embodiments have been described above; however, it should be understood that various modifications may be made. For example, suitable results can be achieved if the described techniques are performed in a different order and/or if components in the described systems, architectures, devices, or circuits are combined in different ways and/or replaced or supplemented by additional components or equivalents thereof. Accordingly, other implementations also fall within the scope of protection of the claims.

1. An image stitching method for an X-ray imaging system comprising an X-ray source, the image stitching method comprising:
dividing an imaging region of a subject under examination into a first region and a second region, the first region and the second region having an overlapping portion;
acquiring a first quantity of X-ray images of the subject under examination by rotating the X-ray source in the first region;
acquiring a second quantity of X-ray images of the subject under examination by translating the X-ray source in the second region; and
performing image stitching on the first quantity of X-ray images and the second quantity of X-ray images to acquire a medical image of the subject under examination.

2. The image stitching method according to claim 1, wherein the dividing an imaging region of a subject under examination into a first region and a second region comprises:
determining a start position, an intermediate position, and an end position of the imaging region to determine the first region and the second region.

3. The image stitching method according to claim 2, wherein a region between the start position and the intermediate position constitutes one of the first region and the second region, a region between a temporary position and the end position constitutes the other of the first region and the second region, the temporary position is a position adjacent to the start position and having a preset distance from the intermediate position, and the overlapping portion is between the temporary position and the intermediate position.

4. The image stitching method according to claim 2, wherein the X-ray imaging system further comprises a camera unit configured to acquire a camera image of the subject under examination, and the determining a start position, an intermediate position, and an end position of the imaging region of the subject under examination comprises determining the start position, the intermediate position, and the end position based on the acquired camera image.

5. The image stitching method according to claim 4, wherein the determining the start position, the intermediate position, and the end position based on the acquired camera image comprises:
performing image processing on the camera image to identify information of an anatomical site or a keypoint of the subject under examination; and
determining the start position, the intermediate position, and the end position based on the information of the anatomical site or the keypoint.

6. The image stitching method according to claim 2, wherein the intermediate position is a position where the pelvis is located.

7. The image stitching method according to claim 1, wherein the first region is a region comprising the spine of the subject under examination, and the second region is a region comprising the lower limbs of the subject under examination.

8. The image stitching method according to claim 1, wherein the acquiring a first quantity of X-ray images of the subject under examination comprises:
dividing the first region into a first quantity of sub-imaging regions;
controlling the X-ray source to respectively rotate by a first quantity of angles to align with the first quantity of sub-imaging regions, respectively; and
acquiring the first quantity of X-ray images corresponding to the first quantity of sub-imaging regions.

9. The image stitching method according to claim 1, wherein the acquiring a second quantity of X-ray images of the subject under examination comprises:
dividing the second region into a second quantity of sub-imaging regions;
controlling the X-ray source to respectively translate to a second quantity of positions to align with the second quantity of sub-imaging regions, respectively; and
acquiring the second quantity of X-ray images corresponding to the second quantity of sub-imaging regions.

10. The image stitching method according to claim 1, wherein the performing image stitching on the first quantity of X-ray images and the second quantity of X-ray images comprises:
performing image stitching on the first quantity of X-ray images to acquire a first region image;
performing image stitching on the second quantity of X-ray images to acquire a second region image; and
stitching the first region image and the second region image to acquire the medical image;
alternatively,
stitching an (m+2)th image acquired from the first quantity of X-ray images or the second quantity of X-ray images and an (m)th temporary image to acquire the medical image, wherein m is an integer greater than or equal to 1, and the (m)th temporary image refers to an intermediate image obtained by stitching the first (m+1) images.

11. An X-ray imaging method, comprising:
determining a first quantity of sub-imaging regions based on a start position and an intermediate position that are set, and determining a second quantity of sub-imaging regions based on the intermediate position and an end position that are set;
controlling the X-ray source to move between the start position and the intermediate position, controlling the X-ray source to rotate to respectively align with the first quantity of sub-imaging regions, and controlling the X-ray source to translate to respectively align with the second quantity of sub-imaging regions, so as to acquire a first quantity of X-ray images and a second quantity of X-ray images, respectively; and
performing image stitching on the first quantity of X-ray images and the second quantity of X-ray images to acquire a medical image.

12. An X-ray imaging system, comprising:
an X-ray source capable of emitting X-rays toward a subject under examination; and
a control unit connected to the X-ray source and capable of controlling the movement of the X-ray source, the control unit comprising:
a region determination unit configured to divide an imaging region of the subject under examination into a first region and a second region, the first region and the second region having an overlapping portion;
a motion control unit configured to acquire a first quantity of X-ray images of the subject under examination by rotating the X-ray source in the first region and to acquire a second quantity of X-ray images of the subject under examination by translating the X-ray source in the second region; and
a stitching unit configured to perform image stitching on the first quantity of X-ray images and the second quantity of X-ray images to acquire a medical image of the subject under examination.
